(19) United States
(12) Patent Application Publication
(10) Pub. No.: US 2024/0391915 A1
(43) Pub. Date: Nov. 28, 2024

(54) CRYSTALLINE FORM OF TOLEBRUTINIB AND PREPARATION METHOD THEREOF

(71) Applicant: Principia Biopharma Inc., Bridgewater, NJ (US)

(72) Inventors: Minhua Chen, Suzhou (CN); Jiaming Shi, Suzhou (CN)

(73) Assignee: Principia Biopharma Inc., Bridgewater, NJ (US)

(21) Appl. No.: 18/676,250

(22) Filed: May 28, 2024

Related U.S. Application Data
(63) Continuation of application No. 17/818,846, filed on Aug. 10, 2022, now Pat. No. 12,049,463.
Application No. 17/818,846 is a continuation of application No. PCT/CN2021/132028, filed on Nov. 22, 2021.

(30) Foreign Application Priority Data
Dec. 10, 2020 (CN) 202011455573.5

(51) Int. Cl. C07D 471/04; A61P 25/00; A61P 37/06

(52) U.S. Cl. CPC C07D 471/04; A61P 25/00; A61P 37/06; C07B 2200/13

(57) ABSTRACT

Disclosed is a crystalline form of Tolebrutinib (hereinafter referred to as “Compound I”) and preparation methods thereof, pharmaceutical compositions containing the crystalline form, and uses of the crystalline form for preparing BTK inhibitor drugs and drugs for treating multiple sclerosis. The provided crystalline form of Tolebrutinib has one or more improved properties and has significant value for future drug optimization and development.

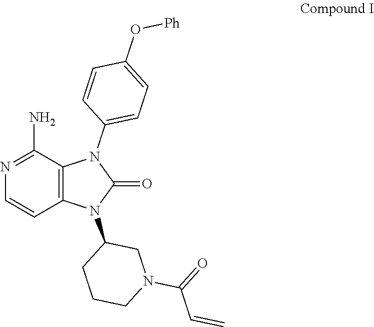 [SMILES: C=CC(=O)N1CCC[C@@H](n2c(=O)n(-c3ccc(Oc4ccccc4)cc3)c3c(N)nccc32)C1]

### CROSS-REFERENCE TO RELATED APPLICATIONS

This application is a continuation of International Application No. PCT/CN2021/132028, filed Nov. 22, 2021, which claims the benefit of priority to Chinese Patent Application 202011455573.5, filed Dec. 10, 2020. The contents of each of the referenced applications are incoroporated herein by reference in their entireties.

### TECHNICAL FIELD

The present disclosure pertains to the field of chemical crystallography, particularly relates to crystalline forms of Tolebrutinib, preparation method and use thereof.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families. MS involves an immune-mediated process in which an abnormal response of the body's immune system is directed against the central nervous system (CNS). In the course of the disease, scleroses, i.e., lesions or scars, appear in the myelin sheath of nerve cells, disrupting transmission of electrical signals. Scleroses accumulate over time and result in the debilitating symptoms experienced by MS patients.

Immunomodulatory drugs have been the mainstay of MS therapy. Results from recent clinical studies have demonstrated efficacy of agents that target B lymphocytes.

The Bruton's tyrosine kinase (BTK) pathway is critical to signaling in B lymphocytes and myeloid cells including CNS microglia. Each of these cell types has been implicated in the pathophysiology of MS. Further, as BTK signaling is vital for maturation of B cells into antibody-secreting plasma cells, BTK inhibition can modulate both cellular and humoral immunity. Accordingly, an inhibitor of BTK signaling represents a dual mechanism targeting both aspects of the immune system.

Accordingly, compounds that inhibit BTK that are able to both inhibit antigen-induced B-cell activation responsible for neuroinflammation and modulate maladaptive microglia cells linked to neuroinflammation in the brain and spinal cord may be useful in treating relapsing multiple sclerosis (RMS) with superior benefits when compared to currently available therapies. Tolebrutinib, an oral selective small-molecule BTK inhibitor, has shown safety and efficacy in patients with RMS.

The chemical name of Tolebrutinib is (R)-1-(1-acryloylpiperidin-3-yl)-4-amino-3-(4-phenoxyphenyl)-1H-imidazo [4,5-c] pyridin-2 (3H)-one (hereinafter referred to as Compound I), and the structure is shown as follows:

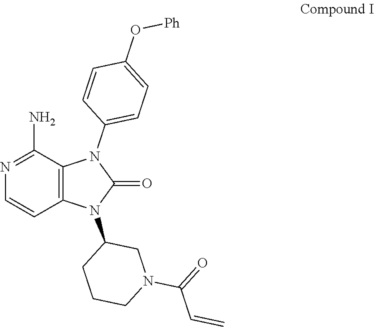 [SMILES: C=CC(=O)N1CCC[C@@H](n2c(=O)n(-c3ccc(Oc4ccccc4)cc3)c3c(N)nccc32)C1]

A crystalline form is a solid material whose constituents are arranged in a highly ordered microscopic structure, forming a crystal lattice that extends in all directions. Polymorphism refers to the phenomenon that a compound exists in more than one crystalline form. Compounds may exist in one or more crystalline forms, but their existence and characteristics cannot be predicted with any certainty. Different crystalline forms of drug substances have different physicochemical properties, which can affect drug's in vivo dissolution and absorption and will further affect drug's clinical efficacy to some extent. In particular, for some poorly soluble oral solid or semi-solid dosage forms, crystalline forms can be crucial to the performance of drug product. In addition, the physiochemical properties of a crystalline form are very important to the production process. Therefore, polymorphism is an important part of drug research and drug quality control.

A white solid of Compound I was disclosed in WO2016196840A1. The inventors of the present disclosure repeated the preparation process and an amorphous of Compound I was obtained. Furthermore, the inventors of the present disclosure have systematically evaluated the properties of the amorphous obtained, and the results show that the amorphous of Compound I has disadvantages such as poor stability, strong hygroscopicity and easy degradability, and is not suitable for medicine use.

In order to overcome the disadvantages of prior arts, the inventors of present disclosure conducted a systematic study on Compound I and found that Compound I easily form amorphous and is difficult to crystallize. Specifically, the inventors of present disclosure designed a large number of experiments including different processing methods, solvent systems and post-treatment processes, trying to obtain a solid form of Compound I with good physicochemical stability, good hygroscopicity and little degradability. While no crystalline form suitable for medicine use was obtained except for amorphous of Compound I. The inventors of present disclosure further tried more methods and surprisingly obtained a crystalline form of Compound I. This crystalline form has advantages in at least one aspect of solubility, hygroscopicity, purification ability, stability, adhesiveness, compressibility, flowability, in vitro and in vivo dissolution, bioavailability, etc. In particular, the crystalline form of Compound I of the present disclosure has advantages such as good stability, good hygroscopicity, and little degradability, which solves the problems existing in the prior art and is of great significance for the development of drugs containing Compound I.

### SUMMARY

The present disclosure is to provide a novel crystalline form of Compound I, preparation method and use thereof.

According to the objective of the present disclosure, the crystalline form of Compound I is provided.

Furthermore, crystalline form CSI of Compound I is provided (hereinafter referred to as Form CSI).

In one aspect provided herein, the X-ray powder diffraction pattern of Form CSI comprises one or two or three characteristic peaks at 2theta values of 7.7°±0.2°, 11.0°±0.2° and 22.8°±0.2° using CuKα radiation. Preferably, the X-ray powder diffraction pattern of Form CSI comprises characteristic peaks at 2theta values of 7.7°±0.2°, 11.0°±0.2° and 22.8°±0.2° using CuKα radiation.

In another aspect provided herein, the X-ray powder diffraction pattern of Form CSI comprises one or two or three characteristic peaks at 2theta values of 12.0°±0.2°, 16.1°±0.2° and 18.5°±0.2° using CuKα radiation. Preferably, the X-ray powder diffraction pattern of Form CSI comprises characteristic peaks at 2theta values of 12.0°±0.2°, 16.1°±0.2° and 18.5°±0.2° using CuKα radiation.

In another aspect provided herein, the X-ray powder diffraction pattern of Form CSI comprises one or two or three characteristic peaks at 2theta values of 13.6°±0.2°, 20.1°±0.2° and 24.8°±0.2° using CuKα radiation. Preferably, the X-ray powder diffraction pattern of Form CSI comprises characteristic peaks at 2theta values of 13.6°±0.2°, 20.1°±0.2° and 24.8°±0.2° using CuKα radiation.

In another aspect provided herein, the X-ray powder diffraction pattern of Form CSI comprises one or two or three or four or five or six or seven or eight or nine characteristic peaks at 2theta values of 7.7°±0.2°, 11.0°±0.2°, 22.8°±0.2°, 12.0°±0.2°, 16.1°±0.2°, 18.5°±0.2°, 13.6°±0.2°, 20.1°±0.2°, 24.8°±0.2° and 18.7°±0.2° using CuKα radiation.

Without any limitation being implied, the X-ray powder diffraction pattern of Form CSI is substantially as depicted in FIG. 2 using CuKα radiation.

Without any limitation being implied, the DSC curve of Form CSI is substantially as depicted in FIG. 6, which shows an endothermic peak at around 170° C. (onset temperature). This peak is the melting endothermic peak.

Without any limitation being implied, the TGA curve of Form CSI is substantially as depicted in FIG. 5, which shows about 0.4% weight loss when heated from 31° C. to 160° C.

Without any limitation being implied, Form CSI is an anhydrate.

According to the objective of the present disclosure, a process for preparing Form CSI is also provided. The process comprises:

Adding the solid of Compound I into an ketone or an ether, stirring at a certain temperature and separating to obtain Form CSI.

Furthermore, said ketone is preferably a ketone of C3-C6, said ether is preferably an ether of C5.

Furthermore, said ketone is preferably 4-methyl-2-pentanone, said ether is preferably methyl tertiary butyl ether.

Furthermore, said stirring temperature is preferably from room temperature to 55° C., said stirring time is preferably more than 25 hours.

According to the objective of the present disclosure, the present disclosure provides the use of Form CSI for preparing other crystalline forms, or salts of Compound I.

According to the objective of the present disclosure, a pharmaceutical composition is provided, said pharmaceutical composition comprises a therapeutically effective amount of the crystalline form of Compound I and pharmaceutically acceptable excipients.

Furthermore, use of the crystalline form of Compound I is provided by present disclosure for the preparation of a BTK inhibitor drug.

Furthermore, use of the crystalline form of Compound I is provided by present disclosure for the preparation of a drug for the treatment of multiple sclerosis.

Furthermore, the crystalline form of Compound I is preferably Form CSI.

### Technical Problems Solved by Present Disclosure

The inventors of the present disclosure studied the prior art and found that the prior art is the amorphous of Compound I. It is found through research that the amorphous of Compound I has disadvantages such as poor chemical stability, poor hygroscopicity and easy degradability, which is not suitable for medicine use and industrial production. In order to overcome the disadvantages of prior arts, a crystalline form of Compound I is provided by the present disclosure, which has excellent physical and chemical stability, good hygroscopicity, and is suitable for the development of drugs containing Compound I.

As shown in Example 1, Compound I is difficult to crystallize. Only amorphous was obtained by various crystallization methods. Even trying different crystallization methods and control the processing conditions in the preparation process, such as: solvent (alcohols, ketones, esters, ethers, acids, water, nitriles, amides, halogenated hydrocarbons, aromatic hydrocarbons, alkanes, sulfoxides, etc.), temperature, time, evaporation rate, additives and other factors, can only obtain amorphous. To obtain Form CSI of the present disclosure, the inventors further tried a variety of unconventional solvents and improved the preparation and post-treatment conditions based on the foregoing preparation methods. This shows that Form CSI provided by present disclosure is unpredictable for the skilled in the art.

### Technical Effects

Form CSI of the present disclosure has the following unexpected advantages:

(1) The chemical purity of the prior art solid decreases significantly when stored under the conditions of 25° C./60% RH, 40° C./75% RH, 60° C./75% RH, and 80° C. In particular, after storage at 40° C./75% RH for 6 months with open package, the purity decreases by 3.46%, and the number of impurities which exceed the qualificated threshold increases to four. After storage at 60° C./75% RH for only 1 month with sealed package, the purity decreases by 2.76%, and the number of impurities which exceed the qualificated threshold increases to two. After storage at 60° C./75% RH for only 1 month with open package, the purity decreases over 6.3%, and the number of impurities which exceed the qualificated threshold increases to four. The chemical stability of the prior art solid is far below the medicinal standard.

Compared with the prior art, Form CSI drug substance of the present disclosure has good stability itself and in drug product. Crystalline state of Form CSI drug substance doesn't change for at least 6 months when stored under the condition of 25° C./60% RH with open and sealed package. The chemical purity is above 99.8% and remains substantially unchanged during storage. After Form CSI is mixed with the excipients to form a drug product and stored under the condition of 25° C./60% RH, crystalline state of Form CSI drug product doesn't change for at least 3 months. These results show that From CSI drug substance of the present disclosure has good stability under long term condition both itself and in drug product which is suitable for drug storage.

Meanwhile, crystalline state of Form CSI drug substance doesn't change for at least 6 months when stored under the condition of 40° C./75% RH with open and sealed package. The crystalline state of Form CSI drug substance doesn't change for at least 1 month when stored under the condition of 60° C./75% RH with open or sealed package. The chemical purity is above 99.8% and remains substantially unchanged during storing. The chemical purity of Form CSI drug substance remains substantially unchanged for at least 2 days when stored under the condition of 80° C. After Form CSI is mixed with the excipients to form a drug product and stored under the condition of 40° C./75% RH, crystalline state of Form CSI drug product doesn't change for at least 3 months. These results show that Form CSI drug substance has better stability under accelerated and stress conditions both itself and in drug product. Drug substance and drug product will go through high temperature and high humidity conditions caused by different season, regional climate and environment during storage, transportation, and manufacturing processes. Therefore, good stability under accelerated and stress conditions is of great importance to the drug development. Form CSI drug substance has good stability under stress conditions both itself and in drug product, which is beneficial to avoid the impact on drug quality due to crystal transformation or decrease in purity during drug storage.

In addition, the impurity content of Form CSI drug substance did not exceed the qualificated threshold throughout the stability investigation processes, which can meet the requirements of pharmaceutical development.

(2) Compared with prior art, Form CSI of the present disclosure has good hygroscopicity. The test results show that the weight gain of Form CSI is only 1/7 that of the prior art. The weight gain of Form CSI at 80% RH is 0.53%, indicating that Form CSI is slightly hygroscopic. The weight gain of the prior art solid at 80% RH is 3.69%, indicating that the prior art is hygroscopic.

In one aspect, poor hygroscopicity tends to cause chemical degradation and polymorph transformation, which directly affects the physical and chemical stability of the drug substance.

In addition, poor hygroscopicity will reduce the flowability of the drug substance, thereby affecting the processing of the drug substance.

In another aspect, drug substance with poor hygroscopicity requires low humidity environment during production and storage, which puts strict requirements on production and imposes higher costs. More importantly, poor hygroscopicity is likely to cause variation in the content of active pharmaceutical ingredients in the drug product, thus affecting drug product quality.

Form CSI provided by the present disclosure with good hygroscopicity is not demanding on the production and storage conditions, which reduces the cost of production, storage and quality control, and has strong economic value.

### BRIEF DESCRIPTION OF THE DRAWINGS

FIG. 1 shows an XRPD pattern of sample 1 according to example 1.

FIG. 2 shows an XRPD pattern of Form CSI according to example 2.

FIG. 3 shows an XRPD pattern of Form CSI according to example 3.

FIG. 4 shows an XRPD pattern of Form CSI according to example 4.

FIG. 5 shows a TGA curve of Form CSI.

FIG. 6 shows a DSC curve of Form CSI.

FIG. 7 shows an XRPD pattern overlay of Form CSI before and after storage (from top to bottom: initial, stored at 25° C./60% RH (open package) for 6 months, stored at 25° C./60% RH (sealed package) for 6 months, stored at 40° C./75% RH (open package) for 6 months, stored at 40° C./75% RH (sealed package) for 6 months, stored at 60° C./75% RH (open package) for 1months, stored at 60° C./75% RH (sealed package) for 1 months).

FIG. 8 shows a DVS plot of Form CSI.

FIG. 9 shows a DVS plot of prior art amorphous.

FIG. 10 shows an XRPD pattern overlay of Form CSI before and after formulation process (from top to bottom: excipients, after formulation process, and Form CSI).

FIG. 11 shows an XRPD pattern overlay of Form CSI drug product stored under different conditions (from top to bottom: initial drug product, stored under 25° C./60% RH for 3 months, stored under 40° C./75% RH for 3 months).

### DETAILED DESCRIPTION

The present disclosure is further illustrated by the following examples which describe the preparation and use of the crystalline forms of the present disclosure in detail. It is obvious to those skilled in the art that changes in the materials and methods can be accomplished without departing from the scope of the present disclosure.

The abbreviations used in the present disclosure are explained as follows:
XRPD: X-ray Powder Diffraction
DSC: Differential Scanning calorimetry
TGA: Thermo Gravimetric Analysis
DVS: Dynamic Vapor Sorption
¹H NMR: Proton Nuclear Magnetic Resonance
RH: Relative humidity
UPLC: Ultra Performance Liquid Chromatography
LC: Liquid Chromatography
PE: Polyethylene
LDPE: Low Density Polyethylene
HDPE: High Density Polyethylene

Instruments and methods used for data collection:

X-ray powder diffraction patterns in the present disclosure were acquired by a Bruker X-ray powder diffractometer. The parameters of the X-ray powder diffraction method of the present disclosure are as follows:
X-Ray: Cu, Kα
Kα1 (Å): 1.54060; Kα2 (Å): 1.54439
Kα2/Kα1 intensity ratio: 0.50

Thermo gravimetric analysis (TGA) data in the present disclosure were acquired by a TA Q500.

The parameters of the TGA method of the present disclosure are as follows:
Heating rate: 10° C./min
Purge gas: nitrogen

Differential scanning calorimetry (DSC) data in the present disclosure were acquired by a TA Q2000. The parameters of the DSC method of the present disclosure are as follows:
Heating rate: 10° C./min
Purge gas: nitrogen

Dynamic Vapor Sorption (DVS) was measured via an SMS (Surface Measurement Systems Ltd.) intrinsic DVS instrument. Typical Parameters for DVS test are as follows:
Temperature: 25° C.
Gas and flow rate: nitrogen, 200 mL/min
RH range: 0% RH to 95% RH

Proton nuclear magnetic resonance spectrum data (¹H NMR) were collected from a Bruker Avance II DMX 400M HZ NMR spectrometer. 1-5 mg of sample was weighed and dissolved in 0.5 mL of deuterated dimethyl sulfoxide to obtain a solution with a concentration of 2-10 mg/mL.

The related substance in the present disclosure was detected by UPLC and the parameters are shown below.

TABLE 1

| Instrument | Waters ACQUITY UPLC H-Class with PDA | |
|---|---|---|
| Column | ACE Excel 3 C18 | |
| Mobile phase | A: 0.1% H₃PO₄ in H₂O (pH4.0, TEA) | |
| | B: Acetonitrile | |
| Gradient | Time (min) | % B |
| | 0.0 | 10 |
| | 0.3 | 10 |
| | 3.5 | 45 |
| | 9.0 | 80 |
| | 11.0 | 80 |
| | 11.1 | 10 |
| | 18.0 | 10 |

| | |
|---|---|
| Run time | 18.0 min |
| Stop time | 0.0 min |
| Injection volume | 1 μL |
| Detector | 226 nm |
| wavelength | |
| Column | 40° C. |
| temperature | |
| Sample | Room temperature |
| temperature | |
| Diluent | 0.01% TFA in Acetonitrile |

In the present disclosure, said “stirring” is accomplished by using a conventional method in the field such as magnetic stirring or mechanical stirring and the stirring speed is 50 to 1800 r/min.

Preferably the magnetic stirring speed is 300 to 900 r/min and mechanical stirring speed is 100to 300 r/min.

Said “separation” is accomplished by using a conventional method in the field such as centrifugation or filtration. The operation of “centrifugation” is as follows: the sample to be separated is placed into the centrifuge tube, and then centrifuged at a rate of 10000 r/min until the solid all sink to the bottom of the tube.

Said “drying” is accomplished by using a conventional method in the field such as vacuum drying, blast drying or free-air drying. The drying temperature can be room temperature or higher. Preferably the drying temperature is from room temperature to about 60° C., or to 50° C., or to 40° C. The drying time can be 2 to 48 hours, or overnight. Drying is accomplished in a fume hood, forced air convection oven or vacuum oven.

Said “room temperature” is not a specific temperature, but a temperature range of 10-30° C.

Said “open packaged” is putting the sample into a glass vial, covering the vial with aluminum foil, and punching 5-10 holes on the foil.

Said “sealed packaged” is putting the sample into a glass vial, capping the vial tightly, and sealing the vial in an aluminum foil bag.

Said “characteristic peak” refers to a representative diffraction peak used to distinguish crystals, which usually can have a deviation of ±0.2° using CuKα radiation.

In the present disclosure, “crystal” or “crystalline form” refers to the crystal or the crystalline form being identified by the X-ray diffraction pattern shown herein. Those skilled in the art are able to understand that the experimental errors depend on the instrument conditions, the sample preparation and the purity of samples. The relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSI of the present disclosure is pure and substantially free of any other crystalline forms. In the present disclosure, the term “substantially free” when used to describe a novel crystalline form, means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

Unless otherwise specified, the following examples were conducted at room temperature.

According to the present disclosure, Compound I and/or its salt used as a raw material is solid (crystalline or amorphous), oil, liquid form or solution. Preferably, Compound I used as a raw material is a solid.

Raw materials of Compound I and/or a salt thereof used in the following examples were prepared by known methods in the prior art, for example, the method disclosed in WO2016196840A1.

### Example 1

### Attempts for Preparing Compound I Solid Form

The inventors of the present disclosure tried various methods and regulated various process conditions for preparing solid forms, such as solvent (alcohols, ketones, esters, ethers, acids, water, nitriles, amides, halogenated hydrocarbons, aromatic hydrocarbons, alkanes, sulfoxides, etc.), temperature, time, evaporation rate, additives, and other factors. More than one hundred experiments were carried out, while only amorphous was obtained. Some of the experimental methods and results are listed in Table 2-6.

TABLE 2

| Methods | Regulated influencing factors | Results |
|---|---|---|
| Stirring | Solvent (alcohols, esters, acids, water, nitriles, aromatic | Amorphous |
| | hydrocarbons, mixtures thereof, etc.), temperature, time | |
| Evaporation | Solvent (alcohols, ketones, esters, ethers, acids, water, | Amorphous |
| | halogenated hydrocarbons, amides, mixtures thereof, | |
| | etc.), additives, time, evaporation rate | |
| Solid vapor | Solvent (alcohols, esters, ethers, water, alkanes, | Amorphous |
| diffusion | amides, sulfoxides, etc.), temperature | |
| Liquid | Solvent (alcohols, ketones, esters, ethers, acids, water, | Amorphous |
| vapor | alkanes, amides, sulfoxides, etc.), time | |
| diffusion | | |
| Summary | Solvent (alcohols, ketones, esters, ethers, acids, water, | Amorphous |
| | nitriles, amides, halogenated hydrocarbons, aromatic | |
| | hydrocarbons, alkanes, sulfoxides, etc.), temperature, | |
| | time, evaporation rate, additives | |

### Method 1

### Stirring

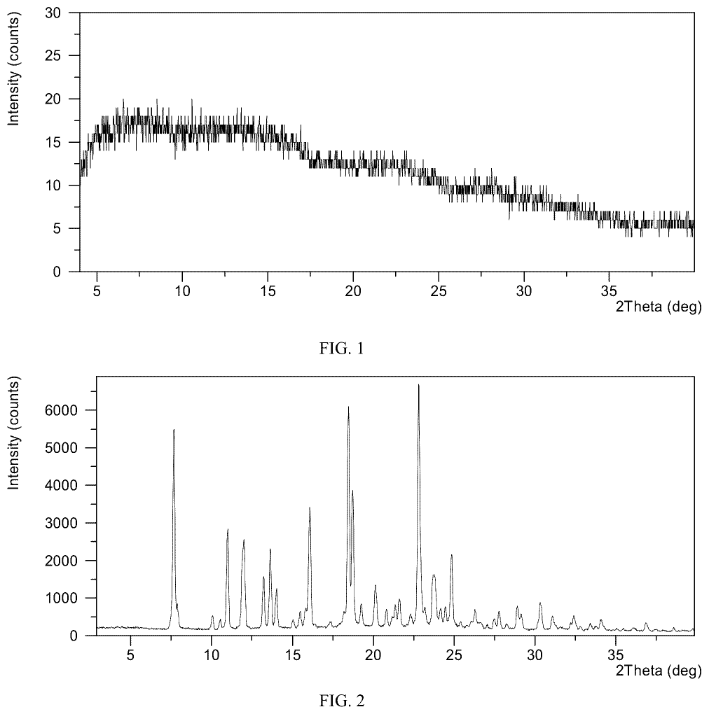

According to Table 3, a certain mass of Compound I solid was weighed into a glass vial, followed by an addition of a certain volume of solvent. After stirring at certain temperature for a period, the solid was separated. All the obtained solids were confirmed to be amorphous by XRPD. The XRPD pattern of sample 1 is substantially as depicted in FIG. 1.

TABLE 3

| | Weight | Solvent | Volume | Temperature | Stirring | |
|---|---|---|---|---|---|---|
| Sample | (mg) | (v/v) | (mL) | (° C.) | time | Solid form |
| 1 | 9.6 | Methanol | 0.2 | Room | 1 day | Amorphous |
| | | | | temperature | | |
| 2 | 8.9 | Ethyl acetate | 0.2 | Room | 1 day | Amorphous |
| | | | | temperature | | |
| 3 | 9.9 | Toluene | 0.2 | Room | 1 day | Amorphous |
| | | | | temperature | | |
| 4 | 9.8 | Water | 0.2 | Room | 1 day | Amorphous |
| | | | | temperature | | |
| 5 | 9.2 | Acetonitrile | 0.2 | Room | 1 day | Amorphous |
| | | | | temperature | | |
| 6 | 19.0 | Isopropyl alcohol | 0.2 | 50 | 1 day | Amorphous |
| 7 | 17.9 | Acetic acid/Water | 0.2 | 50 | 1 day | Amorphous |
| | | (36/64) | | | | |
| 8 | 18.1 | Isopropyl acetate | 0.4 | 50 | 5 days | Amorphous |

### Method 2

### Evaporation

According to Table 4, a certain mass of Compound I solid was weighed into a glass vial. After adding a certain volume of solvent and an additive, the system was evaporated at room temperature. All the obtained solids were confirmed to be amorphous by XRPD.

TABLE 4

| | Weight | Solvent | Volume | | | Evaporation | |
|---|---|---|---|---|---|---|---|
| Sample | (mg) | (v/v) | (mL) | Additive | Time | rate | Solid form |
| 1 | 8.7 | Chloroform | 0.2 | N/A | 4 days | Slow | Amorphous |
| 2 | 8.0 | Tetrahydrofuran | 0.2 | N/A | 4 days | Slow | Amorphous |
| 3 | 8.0 | Ethyl acetate | 0.6 | N/A | 4 days | Slow | Amorphous |
| 4 | 8.5 | Acetone/Water | 1.0 | N/A | 4 days | Slow | Amorphous |
| | | (97/3) | | | | | |
| 5 | 7.6 | Acetone/Water | 1.0 | N/A | 4 days | Slow | Amorphous |
| | | (91/9) | | | | | |
| 6 | 8.0 | N,N-Dimethylformamide/ | 1.0 | N/A | 4 days | Slow | Amorphous |
| | | Water (94/6) | | | | | |
| 7 | 7.6 | Propionic acid | 0.4 | Polyacetal | 1 month | Fast | Amorphous |
| 8 | 8.4 | Tetrahydrofuran/ | 1.6 | Chlorosulfonated | 4 days | Fast | Amorphous |
| | | Methanol (3/2) | | polyethylene | | | |

Fast evaporation: the sample vial is open for evaportation without cap.
Slow evaporation: the sample vial is sealed with cap which has small holes.

### Method 3

### Solid Vapor Diffusion

According to Table 5, a certain mass of Compound I solid was weighed into a glass vial. The vial was put into a larger glass vial containing about 5 mL of corresponding solvent. The larger vial was sealed with a cap and placed at a certain temperature for sufficient contact of solvent atmosphere and the solid in the vial. All the solids were taken out for XRPD test after 1 day and were confirmed to be amorphous.

TABLE 5

| | Weight | | | |
|---|---|---|---|---|
| Sample | (mg) | Solvent | Temperature (° C.) | Solid form |
| 1 | 7.0 | n-Hexane | Room temperature | Amorphous |
| 2 | 8.0 | Water | Room temperature | Amorphous |
| 3 | 7.6 | Dimethyl sulfoxide | Room temperature | Amorphous |
| 4 | 9.9 | N,N- | Room temperature | Amorphous |
| | | Dimethylacetamide | | |
| 5 | 13.0 | Benzyl alcohol | 5 | Amorphous |
| 6 | 9.7 | L-Ethyl lactate | 5 | Amorphous |
| 7 | 13.1 | Petroleum ether | 5 | Amorphous |
| 8 | 11.3 | 1,3-Dioxolane | 5 | Amorphous |

### Method 4

### Liquid Vapor Diffusion

According to Table 6, a certain mass of Compound I solid was weighed into a glass vial and dissolved with a certain volume of solvent. The vial was put into a larger glass vial containing about 5 mL of corresponding anti-solvents, then the larger vial was sealed with a cap and placed at a certain temperature to allow the anti-solvent vapor diffusing into the inner vial sufficiently. All the solids were isolated and confirmed to be amorphous by XRPD after diffusion for different times.

TABLE 6

| | Weight | | Volume | | | |
|---|---|---|---|---|---|---|
| Sample | (mg) | Solvent | (mL) | Anti-solvent | Time | Solid form |
| 1 | 9.8 | Acetic acid | 0.2 | Methanol | 86 days | Amorphous |
| 2 | 10.3 | Acetic acid | 0.3 | Methyl | 86 days | Amorphous |
| | | | | tert-butyl | | |
| | | | | ether | | |
| 3 | 11.0 | Dimethyl sulfoxide | 0.3 | Isopropyl | 86 days | Amorphous |
| | | | | acetate | | |
| 4 | 11.7 | Dimethyl sulfoxide | 0.3 | n-Hexane | 86 days | Amorphous |
| 5 | 9.7 | N -methylpyrrolidone | 0.3 | Methyl | 86 days | Amorphous |
| | | | | isobutyl | | |
| | | | | ketone | | |
| 6 | 10.3 | N,N-dimethylacetamide | 0.3 | Water | 1 day | Amorphous |
| 7 | 11.8 | N, | 0.3 | Water | 9 days | Amorphous |
| | | N-dimethylformamide | | | | |
| 8 | 12.4 | N, | 0.3 | Methyl | 86 days | Amorphous |
| | | N-dimethylformamide | | tert-butyl | | |
| | | | | ether | | |

The above experimental results indicate that Compound I is difficult to crystallize and amorphous is easily obtained. The inventors of the present disclosure further tried various unconventional solvents and improved the preparation and post-treatment conditions, as described in Example 2-4, and the crystal form of Compound I was finally obtained unexpectedly.

### Example 2

### Preparation Method of Form CSI

300.8 mg of Compound I solid was weighed into a 3-mL glass vial, followed by the addition of 2.0 mL of methyl isobutyl ketone. After stirring at 50° C. for about 39 hours, a solid was isolated. The obtained solid is confirmed to be Form CSI of the present disclosure. The XRPD pattern is substantially as depicted in FIG. 2, and the XRPD data are listed in Table 7.

TABLE 7

| | | Relative |
|---|---|---|
| 2θ (°) | d spacing (Å) | intensity (%) |
| 7.69 | 11.50 | 81.95 |
| 7.89 | 11.21 | 10.33 |
| 10.07 | 8.78 | 5.49 |
| 10.55 | 8.39 | 4.24 |
| 11.00 | 8.04 | 41.35 |
| 11.88 | 7.45 | 26.12 |
| 12.04 | 7.35 | 34.15 |
| 13.22 | 6.70 | 21.79 |
| 13.64 | 6.49 | 33.48 |
| 14.02 | 6.32 | 16.90 |
| 15.02 | 5.90 | 3.83 |
| 15.47 | 5.73 | 7.44 |
| 15.80 | 5.61 | 8.05 |
| 16.10 | 5.51 | 47.60 |
| 17.36 | 5.11 | 3.12 |
| 18.47 | 4.80 | 92.44 |
| 18.73 | 4.74 | 57.54 |
| 19.24 | 4.61 | 10.58 |
| 20.14 | 4.41 | 18.59 |
| 20.83 | 4.26 | 8.44 |
| 21.36 | 4.16 | 10.50 |
| 21.62 | 4.11 | 12.80 |
| 22.30 | 3.99 | 6.58 |
| 22.79 | 3.90 | 100.00 |
| 23.66 | 3.76 | 21.01 |
| 23.83 | 3.73 | 21.02 |
| 24.18 | 3.68 | 8.80 |
| 24.46 | 3.64 | 9.62 |
| 24.85 | 3.58 | 31.33 |
| 26.29 | 3.39 | 8.42 |
| 27.45 | 3.25 | 4.21 |
| 27.77 | 3.21 | 7.89 |
| 28.25 | 3.16 | 2.45 |
| 28.89 | 3.09 | 9.70 |
| 29.14 | 3.06 | 6.68 |
| 30.32 | 2.95 | 11.30 |
| 31.09 | 2.88 | 6.06 |
| 32.41 | 2.76 | 5.91 |
| 33.41 | 2.68 | 2.46 |
| 34.08 | 2.63 | 4.17 |
| 36.11 | 2.49 | 1.14 |
| 36.84 | 2.44 | 3.09 |

### Example 3

### Preparation Method of Form CSI

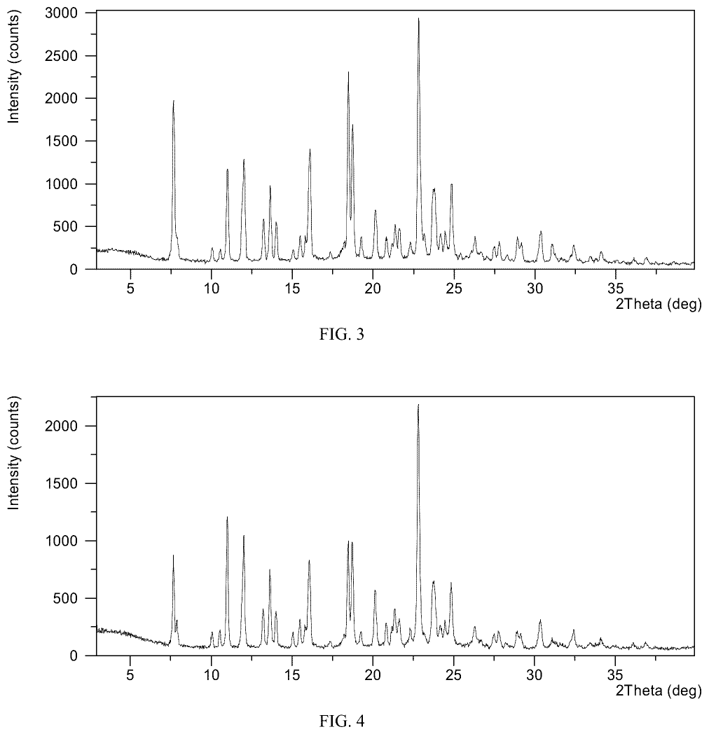

300.1 mg of Compound I solid was weighed into a 3-mL glass vial, followed by the addition of 2.0 mL of methyl isobutyl ketone. After stirring at 50° C. for about 6 days, a solid was isolated. The obtained solid is confirmed to be Form CSI of the present disclosure by XRPD. The XRPD pattern is substantially as depicted in FIG. 3, and the XRPD data are listed in Table 8.

TABLE 8

| | | Relative |
|---|---|---|
| 2θ (°) | d spacing (Å) | intensity (%) |
| 7.64 | 11.57 | 64.68 |
| 10.06 | 8.79 | 5.16 |
| 10.53 | 8.40 | 4.30 |
| 10.98 | 8.06 | 37.82 |
| 12.03 | 7.36 | 40.10 |
| 13.21 | 6.70 | 17.22 |
| 13.63 | 6.50 | 31.16 |
| 14.00 | 6.32 | 16.13 |
| 15.04 | 5.89 | 4.71 |
| 15.47 | 5.73 | 10.37 |
| 15.79 | 5.61 | 10.80 |
| 16.09 | 5.51 | 46.03 |
| 17.36 | 5.11 | 3.85 |
| 18.46 | 4.81 | 76.52 |
| 18.73 | 4.74 | 56.08 |
| 19.26 | 4.61 | 10.27 |
| 20.12 | 4.41 | 21.12 |
| 20.81 | 4.27 | 10.44 |
| 21.36 | 4.16 | 15.59 |
| 21.63 | 4.11 | 13.59 |
| 22.29 | 3.99 | 7.89 |
| 22.79 | 3.90 | 100.00 |
| 23.68 | 3.76 | 27.31 |
| 23.82 | 3.74 | 29.20 |
| 24.15 | 3.69 | 11.27 |
| 24.44 | 3.64 | 12.52 |
| 24.83 | 3.59 | 32.01 |
| 26.31 | 3.39 | 10.77 |
| 27.43 | 3.25 | 5.57 |
| 27.77 | 3.21 | 7.24 |
| 28.27 | 3.16 | 2.98 |
| 28.91 | 3.09 | 10.74 |
| 29.18 | 3.06 | 7.70 |
| 30.39 | 2.94 | 12.58 |
| 31.04 | 2.88 | 7.39 |
| 32.42 | 2.76 | 7.07 |
| 33.44 | 2.68 | 2.63 |
| 34.03 | 2.63 | 3.54 |
| 36.13 | 2.49 | 1.61 |
| 36.85 | 2.44 | 2.20 |

### Example 4

### Preparation of Form CSI

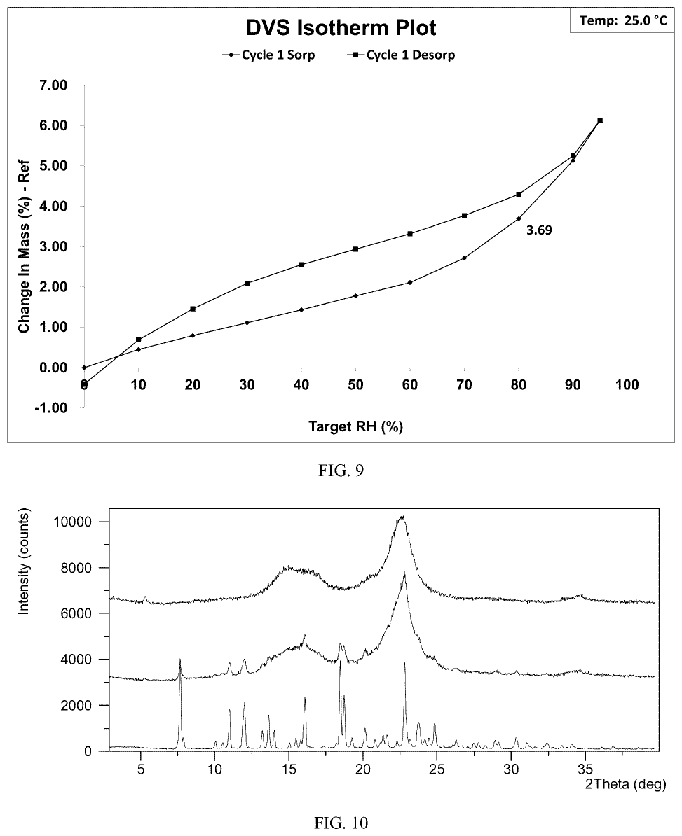

300.4 mg of Compound I solid was weighed into a glass vial, followed by the addition of 3.0 mL of methyl tert-butyl ether. After stirring at 50° C. for about 68 hours, a solid was isolated. After vacuum drying at 75° C. for 1 hour, the obtained solid is confirmed to be Form CSI of the present disclosure, and the XRPD data are shown in FIG. 9 and Table 4.

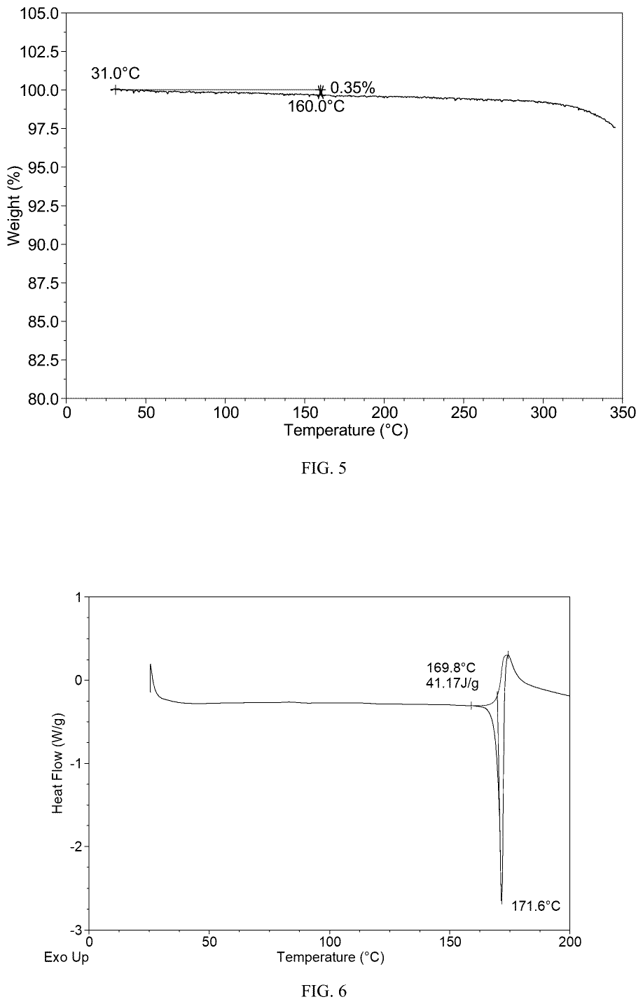

The TGA curve is substantially as depicted in FIG. 5, which shows about 0.4% weight loss when heated from 31° C. to 160° C.

The DSC curve is substantially as depicted in FIG. 6. It shows one endothermic peak at around 170° C. (onset temperature), which is the melting endothermic peak of Form CSI.

The ¹H NMR data are as follows: ¹HNMR (400 MHZ, DMSO) δ (ppm) 7.75 (d, 1H), 7.52-7.36 (m, 4H), 7.21 (t, 1H), 7.14 (t, J-7.8 Hz, 4H), 6.98 (d, 1H), 6.91-6.76 (m, 1H), 6.13 (dd, J=16.5, 7.0 Hz, 1H), 5.69 (dd, J=16.7, 10.8 Hz, 1H), 4.82 (s, 2H), 4.50 (t, J-14.3 Hz, 1H), 4.15 (dd, J-33.9, 12.5 Hz, 2H), 3.76 (t, J=13.0 Hz, 0.5H), 3.16 (t, J=12.7 Hz, 0.5H), 2.79-2.61 (m, 0.5H), 2.45-2.29 (m, J=13.0, 9.1 Hz, 1H), 2.10-1.74 (m, 2H), 1.66-1.37 (m, 1H). (According to the structure of Compound I, the peak of one piperidine hydrogen appears at 8 3.33-3.76 ppm. Harf of this hydrogen is spitted and covered by the signal of water since it is close to the peak of water.)

TABLE 9

| | | Relative |
|---|---|---|
| 2θ (°) | d spacing (Å) | intensity (%) |
| 7.67 | 11.53 | 36.95 |
| 7.88 | 11.22 | 10.54 |
| 10.07 | 8.78 | 6.04 |
| 10.56 | 8.38 | 6.47 |
| 11.00 | 8.04 | 54.02 |
| 12.03 | 7.36 | 45.91 |
| 13.21 | 6.70 | 15.50 |
| 13.63 | 6.50 | 32.17 |
| 14.02 | 6.32 | 14.97 |
| 15.06 | 5.88 | 6.22 |
| 15.49 | 5.72 | 11.71 |
| 16.08 | 5.51 | 35.68 |
| 17.33 | 5.12 | 2.04 |
| 18.47 | 4.80 | 42.70 |
| 18.73 | 4.74 | 43.49 |
| 19.26 | 4.61 | 6.38 |
| 20.11 | 4.42 | 22.02 |
| 20.85 | 4.26 | 9.97 |
| 21.34 | 4.16 | 15.95 |
| 21.65 | 4.11 | 12.00 |
| 22.32 | 3.98 | 7.77 |
| 22.79 | 3.90 | 100.00 |
| 23.68 | 3.76 | 24.11 |
| 24.18 | 3.68 | 8.70 |
| 24.46 | 3.64 | 11.52 |
| 24.82 | 3.59 | 23.38 |
| 26.30 | 3.39 | 9.10 |
| 27.49 | 3.25 | 5.45 |
| 27.79 | 3.21 | 6.74 |
| 28.90 | 3.09 | 6.79 |
| 30.38 | 2.94 | 11.72 |
| 31.14 | 2.87 | 2.74 |
| 32.42 | 2.76 | 7.51 |
| 34.13 | 2.63 | 3.52 |
| 36.12 | 2.49 | 1.82 |
| 36.90 | 2.44 | 2.04 |

### Example 5

### Physical and Chemical Stability of Form CSI

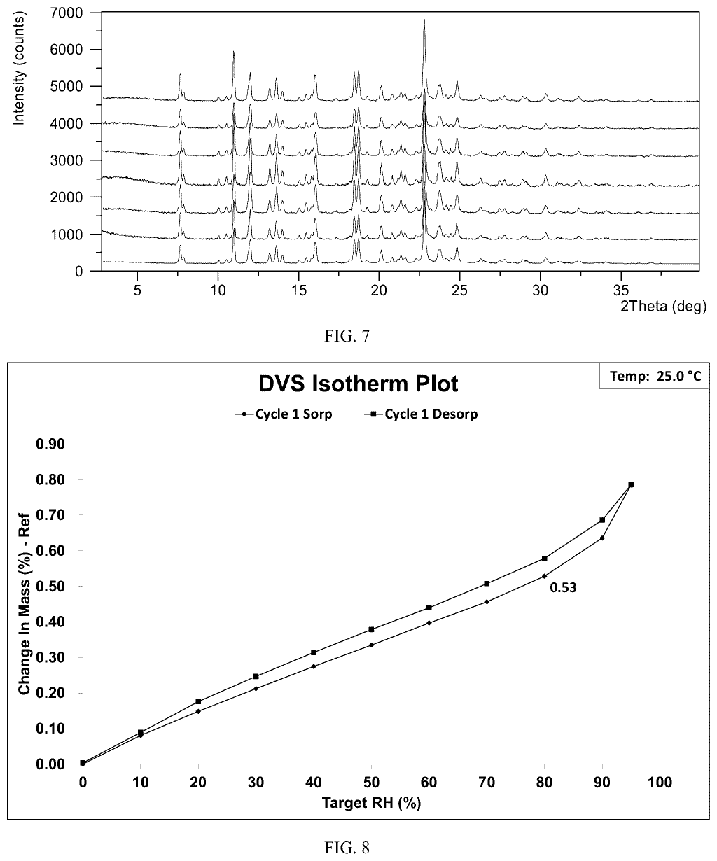

A certain amount of Form CSI of the present disclosure and prior art amorphous were weighed and stored under 25° C./60% RH, 40° C./75% RH and 60° C./75% RH conditions, respectively. The purity and solid form were determined by UPLC and XRPD. The results are listed in Table 10, and the XRPD overlay of Form CSI before and after stability evaluation is shown in FIG. 7.

TABLE 10

| | | | | | | | Impurity |
|---|---|---|---|---|---|---|---|
| | | | | | | | number |
| | | | | | | | exceed the |
| Initial solid | Storage | Packing | Storage | Solid | | Purity | qualificated |
| form | condition | condition | time | form | Purity | change | threshold |
| Form CSI | Initial | N/A | N/A | Form CSI | 99.86% | N/A | 0 |
| | 25° C./60% RH | Sealed | 6 months | Form CSI | 99.89% | 0.03% | 0 |
| | | packaged | | | | | |
| | 25° C./60% RH | Open | 6 months | Form CSI | 99.81% | −0.05% | 0 |
| | | packaged | | | | | |
| | 40° C./75% RH | Sealed | 6 months | Form CSI | 99.92% | 0.06% | 0 |
| | | packaged | | | | | |
| | 40° C./75% RH | Open | 6 months | Form CSI | 99.81% | −0.05% | 0 |
| | | packaged | | | | | |
| | 60° C./75% RH | Sealed | 1 month | Form CSI | 99.85% | −0.01% | 0 |
| | | packaged | | | | | |
| | 60° C./75% RH | Open | 1 month | Form CSI | 99.86% | 0.00% | 0 |
| | | packaged | | | | | |
| Amorphous | Initial | N/A | N/A | Amorphous | 99.80% | N/A | 1 |
| | 25° C./60% RH | Sealed | 6 months | Amorphous | 99.65% | −0.15% | 1 |
| | | packaged | | | | | |
| | 25° C./60% RH | Open | 6 months | Amorphous | 99.57% | −0.23% | 1 |
| | | packaged | | | | | |
| | 40° C./75% RH | Sealed | 6 months | Amorphous | 99.18% | −0.62% | 2 |
| | | packaged | | | | | |
| | 40° C./75% RH | Open | 6 months | Amorphous | 96.34% | −3.46% | 4 |
| | | packaged | | | | | |
| | 60° C./75% RH | Sealed | 1 month | Amorphous | 97.04% | −2.76% | 2 |
| | | packaged | | | | | |
| | 60° C./75% RH | Open | 1 month | Amorphous | 93.48% | −6.32% | 4 |
| | | packaged | | | | | |

The dose of Compound I is 60 mg once daily.

The results show that Form CSI is stable for at least 6 months under 25° C./60% RH and 40° C./75% RH conditions, and the solid form and purity remain basically unchanged, indicating Form CSI has good stability under both long-term and accelerated conditions. After storage under 60° C./75% RH condition for 1 month, the solid form and purity remain basically unchanged, indicating Form CSI has good stability under stressed condition as well. The impurity content of Form CSI drug substance does not exceed the qualificated threshold throughout the stability investigation processes, which meets the requirements of pharmaceutical development. After storage at 25° C./60% RH, 40° C./75% RH and 60° C./75% RH, the purity of prior art amorphous decreased significantly, which is far below the requirements of pharmaceutical development. After storage at 40° C./75% RH for 6 months with open package, the purity decreased by 3.46%, and the number of impurities exceeding the qualificated threshold increased to four. After storage at 60° C./75% RH for only 1 month with sealed package, the purity decreased by 2.76%, and the number of impurities exceeding the qualificated threshold increased to two. After storage at 60° C./75% RH for only 1 month with open package, the purity decreased over 6.3%, and the number of impurities exceeding the qualificated threshold increased to four. The results indicate that Form CSI of the present disclosure has outstanding chemical stability when compared with prior art amorphous.

### Example 6

### Stability of Form CSI at High Temperature

Approximately 10 mg of Form CSI of the present disclosure and prior art amorphous were stored at 80° C. for 2 days, and the initial and final purities were determined by UPLC, as shown in Table 11.

TABLE 11

| Initial | | Storage | Initial | Final | Purity |
|---|---|---|---|---|---|
| solid form | Package condition | time | purity | purity | change |
| Form CSI | Glass vial with cap | 2 days | 99.94% | 99.95% | 0.01% |
| Amorphous | Glass vial with cap | 2 days | 99.80% | 98.64% | −1.16% |

The results indicate that the chemical purity of Form CSI basically remains unchanged for 2 days at 80° C., while significant degradation of amorphous is observed under the same condition. Form CSI of the present disclosure has superior stability at high temperature compared with the prior art amorphous.

### Example 7

### Hygroscopicity of Form CSI

Certain amounts of Form CSI of the present disclosure and prior art amorphous were sampled for hygroscopicity tests using dynamic vapor sorption (DVS) instrument. The weight change at each relative humidity is recorded during the cycle of 0% RH-95% RH-0% RH at 25° C., and the experimental results are listed in Table 12. The DVS plots of Form CSI and amorphous are as depicted in FIG. 8 and FIG. 9, respectively.

TABLE 12

| | Weight gain |
|---|---|
| Form | at 80% RH |
| Form CSI | 0.53% |
| Prior art solid | 3.69% |

The results show that Form CSI is slightly hygroscopic with a weight gain of 0.53% at 80% RH, while prior art solid is hygroscopic with a weight gain of 3.69% at 80% RH. The hygroscopicity of Form CSI is superior to that of prior art.

Description and definition of hygroscopicity (general notice 9103 drug hygroscopicity test guidelines in 2020 edition of Chinese Pharmacopoeia, experimental condition: 25±1° C., 80±2% RH):

Deliquescent: sufficient water is absorbed to form a liquid.

Very hygroscopic: increase in mass is equal to or greater than 15.0 percent.

Hygroscopic: increase in mass is less than 15.0 percent and equal to or greater than 2.0 percent.

Slightly hygroscopic: increase in mass is less than 2.0 percent and equal to or greater than 0.2percent.

Non hygroscopic or almost non hygroscopic: increase in mass is less than 0.2 percent.

(The definition of hygroscopicity in the 10^{th} European Pharmacopoeia 5.11 is similar to the Chinese Pharmacopoeia.)

### Example 8

### Preparation of Form CSI Drug Product

According to the formulation and process in Table 13 and Table 14, the drug products were prepared with an appropriate amount of Form CSI of the present disclosure. XRPD were tested before and after formulation. The XRPD overlay is shown in FIG. 10, indicating Form CSI of the present disclosure is physically stable before and after formulation process.

TABLE 13

| No. | Component | mg/unit | % (w/w) | Function |
|---|---|---|---|---|
| 1 | Form CSI | 20 | 20 | API |
| 2 | Microcrystalline Cellulose | 69.5 | 69.5 | Fillers |
| 3 | Hydroxypropyl | 3.0 | 3.0 | Adhesives |
| | methyl cellulose | | | |
| 4 | Crospovidone | 6.0 | 6.0 | Disintegrants |
| 5 | Colloidal silicon dioxide | 0.5 | 0.5 | Glidants |
| 6 | Magnesium stearate | 1.0 | 1.0 | Lubricants |

| | | | |
|---|---|---|---|
| Total | 100.0 | 100.0 | / |

TABLE 14

| Stage | Procedure |
|---|---|
| Pre-blending | According to the formulation, No. 1-6 materials were weighed into a LDPE |
| | bag and blended for 2 minutes. |
| Simulation of | The pre-mixed powders were tableted by the ENERPAC single punch manual |
| dry granulation | tablet press equipped with a round die of φ 20 mm (tablet weight: 500 ± |
| | 100 mg; pressure: 5 ± 1 KN). The obtained tablets were pulverized and sieved |
| | through a 20-mesh sieve, and then the final mixed powders were obtained. |
| Tableting | The final mixed powders were tableted by the ENERPAC single punch |
| | manual tablet press equipped with a die of φ 9 * 4 mm (tablet weight: 100 ± |
| | 10 mg; pressure: 5 ± 1 KN). |
| Packing | one tablet of drug product and 1 g of desiccant were placed in a sealed 35 cc |
| | HDPE bottle. |

### Example 9

### The Stability of Form CSI Drug Product

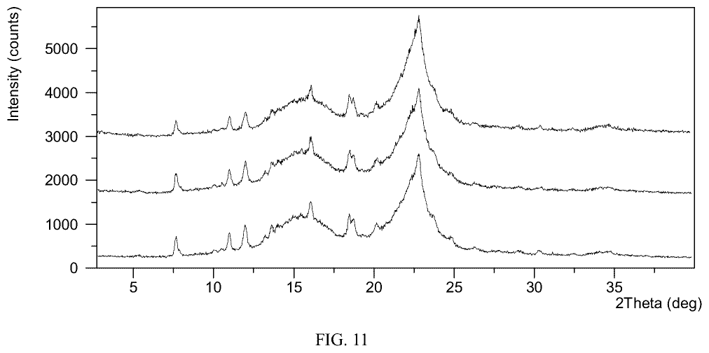

To evaluate the stability of Form CSI in drug products, the packaged drug products prepared in Example 8 were stored under 25° C./60% RH and 40° C./75% RH conditions for 3 months, and the XRPD overlay of drug products before and after storage is as depicted in FIG. 11.

The results indicate that the drug products of Form CSI are stable under 25° C./60% RH and 40° C./75% RH conditions for at least 3 months.

The examples described above are only for illustrating the technical concepts and features of the present disclosure and intended to make those skilled in the art being able to understand the present disclosure and thereby implement it and should not be concluded to limit the protective scope of this disclosure. Any equivalent variations or modifications according to the spirit of the present disclosure should be covered by the protective scope of the present disclosure.

1. A crystalline form of Compound I, wherein the X-ray powder diffraction pattern comprises at least one characteristic peak at 2theta values of 7.7°±0.2°, 11.0°±0.2°, and 22.8°±0.2° using CuKα radiation

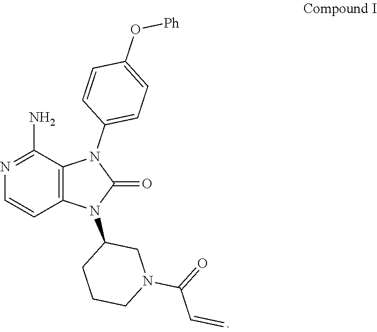 [SMILES: C=CC(=O)N1CCC[C@@H](n2c(=O)n(-c3ccc(Oc4ccccc4)cc3)c3c(N)nccc32)C1]

2. The crystalline form of Compound I according to claim 1, wherein the X-ray powder diffraction pattern comprises at least one characteristic peak at 2theta values of 12.0°±0.2°, 16.1°±0.2°, and 18.5°±0.2° using CuKα radiation.

3. The crystalline form of Compound I according to claim 1, wherein the X-ray powder diffraction pattern comprises at least one characteristic peak at 2theta values of 13.6°±0.2°, 20.1°±0.2°, and 24.8°±0.2° using CuKα radiation.

4. The crystalline form of Compound I according to claim 1, wherein the X-ray powder diffraction pattern is substantially as depicted in FIG. 2 using CuKα radiation.

5. A process for preparing the crystalline form according to claim 1, wherein the process comprises: adding the solid of Compound I into a ketone or an ether, stirring and separating to obtain the crystalline form.

6. The process according to claim 5, wherein said ketone is a ketone of C3-C6, and said ether is an ether of C5.

7. The process according to claim 5, wherein said ketone is 4-methyl-2-pentanone, and said ether is methyl tert-butyl ether.

8. The process according to claim 5, wherein a temperature of stirring is from room temperature to 55° C., and a time of stirring is more than 25 hours.

9. A pharmaceutical composition, wherein said pharmaceutical composition comprises a therapeutically effective amount of the crystalline form according to claim 1, and pharmaceutically acceptable excipients.

10. A method of inhibiting BTK, comprising administering to a subject in need thereof a therapeutically effective amount of the crystalline form according to claim 1.

11. A method of treating multiple sclerosis, comprising administering to a subject in need thereof a therapeutically effective amount of the crystalline form according to claim 1.
